(19) United States
(12) Patent Application Publication
(10) Pub. No.: US 2009/0105207 A1
(43) Pub. Date: Apr. 23, 2009

(54) Copper (1) Complex

(76) Inventors: Salah Fathi Hussein, Cairo (EG); Mohamed Abd El Rahman Sidahmed Goher, Alexandria (EG); Lobna Anwar Abdel Tawwad Abo Bakr, (US)

Correspondence Address: Mohamed Abd El Rahman Sidahmed Goher;c/o Barry G. Magidoff, GREENBERG TRAURIG, LLP, 200 Park Avenue, New York, NY, 10166, US

(21) Appl. No.: 12/238,001

(22) Filed: Sep. 25, 2008

(30) Foreign Application Priority Data
Jun. 25, 2006 (EG) PCT/EG2006/000024
Sep. 21, 2008 (EG) PCT/EG2008/000034

(51) Int. Cl. A61K 31/555; C07F 15/00; A61P 21/00

(52) U.S. Cl. 514/184; 544/225

(57) ABSTRACT

Copper (I) chloride complex of pyrazinic acid is prepared, characterized by elemental analysis, IR, UV-visible spectra and its crystal structure determined by single crystal diffraction methods. This compound is found to exhibit a very positive influence as a drug, in a pharmaceutical acceptable composition for different incurable diseases, e.g. myopathy or weakness of muscles in general, infertility, and certain genetically causes disorders in mammals. These products were found to improve symptoms of myopathy, male infertility, fatigue and weakness of muscles for different reasons, and certain genetically causes disorders in mammals. It also enhanced regeneration of tissue affected by skin burns, scars, improved alopecia, decreased blood lipids and helped in loss of weight in obese patients. The observed wide scope of activities probably due to stem cell stimulation, enhancement the immune system and role of copper dependent enzyme activity.

### BACKGROUND

Duchene dystrophy (DD) is an x linked disorder primarily affecting skeletal muscle, caused by lack of dystrophin—the protein product of DD gene located on XP21. These individuals are males who suffer from progressive muscle weakness extending to both cardiac and respiratory muscle failure. DD suffers die at an early age of either respiratory and/or cardiac failures. Females are healthy carriers. Several prior trials for both drug and genetic treatment did not give satisfactory results.

Male infertility is a multi factorial disease process with a number of potential contributing causes. Considering the majority of male infertility cases are due to deficient sperm production of unknown origin, environmental and mutational factors must be evaluated.

The treatment of male factor infertility is a rapidly developing field. Introduction of microsurgical fertilization techniques allows assisted conception units to treat couples who previously would not have benefited from in vitro fertilization techniques. However these techniques are only used for the minority of sub-fertile men in andrological practice. Many subfertile men are still treated pharmacologically or by sperm selection methods to enhance sperm fertilization ability. Numerous pharmacological compound have been described that enhance sperm motility and thus potentially sperm fertilizing capacity. Sperm motility plays an important role in the normal fertilization process. Poor sperm motility (<50% motile sperm with <2+forward progression according to WHO protocol) is considered a major factor in diminished rates of fertilization Medical trials for male non obstructive infertility by hormonal replacement, corticosteroids (in immunes infertility), mutational therapies but results were not satisfactory. Chronic fatigue is world wide complaint affecting the productivity of a good percentage of the population. Chronic fatigue includes unexplained fatigue, chronic fatigue syndrome and fibromyalgia. In all types of fatigue the complex relieved the muscle pain, increased the physical activity and productivity and associated depression.

Cardiomyopathy is this fatal disease where all clinical trials are just symptomatic treatment for heart failure and the hope for these patients are cardiac transplantation

### DISCLOSURE OF THE INVENTION

According to one broad form of the invention there is provided a process for the preparation of pyrazinic acid-copper halide (I) complex by dissolving the components in a polar solvent e.g. acetone, water or ethanol, to form a bright red microcrystalline powder precipitate. The chloride (I) is the preferred halide.

According to another broad form of the invention there is provided a method which comprises administering to a mammal, e.g. a human, an effective amount of the complex in a pharmaceutically acceptable composition with other acceptable vitamins, carriers, diluent and or excipient. The concentration of the complex and other components depends on different factors, e.g. type and reason of weakness of the muscles, age of the patient, etc.

According to another broad form of the invention there is provided a method for treatment of fatigue. The method comprises administering to an individual, e.g. human, a copper chloride-pyrazinic acid complex and/or a pharmaceutically acceptable composition of the complex. The concentration of the complex in the pharmaceutically acceptable form is variable and depends on several factors, e.g. age of the patient, reason of fatigue, etc. According to a further broad form of the invention there is provided a method for treatment of infertility in men. The method comprises administering to an individual, e.g. a man, the pyrazinic acid complex or a pharmaceutically acceptable composition containing the complex, with an acceptable carrier, diluent or excipient.

Solid dosage forms for oral administration may include capsules, tablets, pills and granules. In such solid dosage forms, the pyrazinic acid-copper chloride complex may be admixed with at least one inert diluent.

Such dosage forms may also comprise, as is normal practice, additional substances other than inert diluents, e.g. ascorbic acid, other vitamins, etc. In the case of capsules the dosage forms may also comprise buffering agents.

### GENERAL DESCRIPTION OF THE DRAWING

FIG. 1 shows the asymmetric form of the Copper Complex, along with the labeling.

### BEST MODE AND OTHER MODES FOR CARRYING OUT THE INVENTION

An effective amount of a copper chloride-pyrazinic acid complex, to achieve a desired level of improving fatigue, infertility, weakness of muscles, etc., can be administered orally to an individual, e.g., a human person. The composition for this purpose is presented as, e.g., capsules or tablets. The specific dose level for a particular person depends on a variety of factors including age, general health, sex, diet, body weight, time of administration.

1. Preparation, Physical Properties and Crystal Structure of [CuCI (pyrazinic acid)₂.2H2O Complex

A. Preparation of the Complex

A suspension of copper (I) chloride in aqueous/acetone solution (1:1 v/v) was added drop-wise to pyrazinic acid (31 g) dissolved in boiled acetone (1 L) with continuous boiling and stirring until a clear reddish solution mixture obtained. The final mixture was cooled and allowed to stand over several hours to deposit a red microcrystalline precipitate of the complex. The precipitate was filtered off at the pump and washed several times with acetone and dried in vacuum. Yield is about 65%. An advantage of this method is that it can be used to prepare the coplex in quantities sufficient for industrial purposes.

Analytical data: Found: C, 31.1; H, 3.0; N, 14.0; Cl, 9.7; Cu, 16.6%. Calculated for C10H12 N4CuCl: C 31.2; H 3.2; N 14.1; Cl 9.3 Cu 16.6%.

B. Physical Properties and Crystal Structure:

Color: bright red-brown microcrystalline powder Stability: the complex is sufficiently stable when well-dried

Solubility: the complex is insoluble in non polar solvents, e.g. benzene, carbon tetrachloride, etc., and insoluble in polar solvents: water, methanol, ethanol, acetone, but soluble in these solvents upon heating in inert atmosphere, to prevent oxidation.

UV-visible spectra: Solid sample mulled in Nujol shows an absorption band due to Cu-L Charge Transfer transition at 425 nm.

Infrared spectrum: exhibits &#957; C═O band around 1700 cm⁻¹, and &#957; C—O around 1350 cm⁻¹ (KBr pellets) The composition of the complex formulated as [CuCI (pyrazinic acid) 2]. 2H2O was confirmed by the single crystal diffraction.

X-ray crystallography: empirical formula: C10H12ClCuN4O6, formula weight: 383.24, Orthorhombic, space group P21212, unit cell dimensions: a=30.693(6)&#506; b=3.6405 (7) &Aring;, c=6.0918(12) &Aring;, alpha=90&#730; beta=90&#730; gamma=

90&#730; volume=680.7(2) &#506; Z=2. Density, calc.=1.837 itim-1. The X-ray single crystal data were collected on a modified STOE four-circle diffract meter. Crystal size 0.35×0.24×0.15 mm. Graphite mono achromatized Mo K&#945; radiation (&#955;=0.71069 &Aring;) with the w scan technique was used to collect the data set. The accurate unit cell parameters were determined and refined by least-squares method. The structure was solved by direct methods and refined by full-matrix least squares methods on F2, using SHELXTL/PC V 5.03 program package. Goodness of fit on the F2 is 1.258. Maximum and minimum peaks in the final difference Fourier synthesis were 1.030 and −1.141 e&#506;-3.

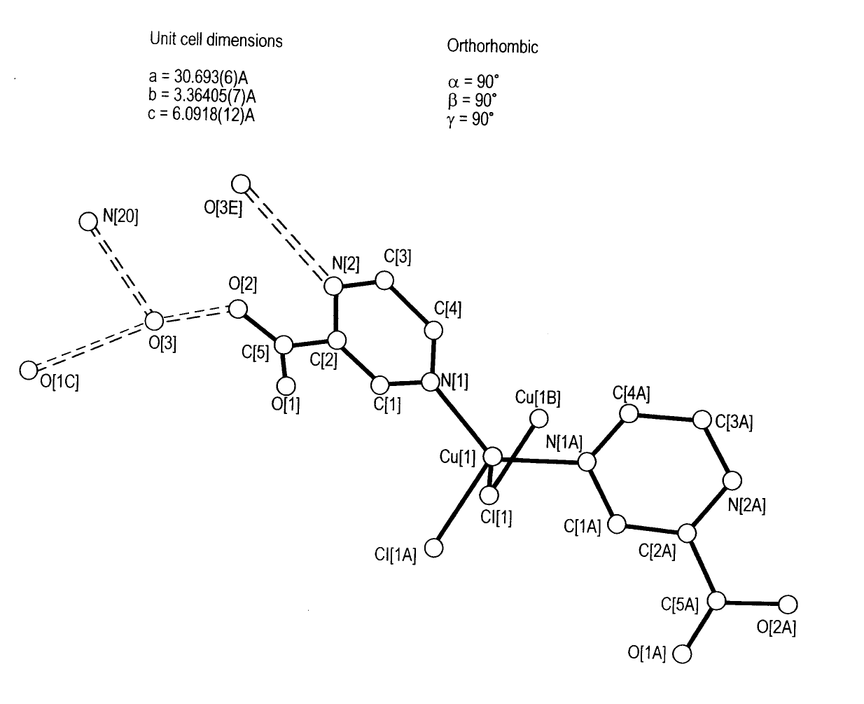

The attached FIG. 1 shows the asymmetric unit along with the labeling scheme.

2. Toxicity of the Complex

Seventy two rats (250+/−2.0 g b.wt) were randomly located into 12 groups of 6 rats each

Rats were observed for any toxic symptoms or signs of discomfort. Mortality in each group were recorded within 24 hrs. The LD50 was calculated according to Behecens and Karbere (1970)

LD50 (I. P.) (oral)=1104.17 mg/kg

LD50 (intrapperitoneally) 128 mg/Kg

3. Bioavailability Studies on Rats

The study on rats included five groups, group 1 control, 2 receiving cu, group.2 pyrazinic, group 3 complex with large dose, & group 5 receiving complex with small dose. There was no significant difference between control groups and other groups regarding the liver and, kidney functions. Complete blood picture were done for all groups, groups 4 and 5 receiving the complex showed increase of lymphocytic count, but not exceeding the normal range suggesting its role in immune function. Lipid profile for all the groups didn't show significant difference.

4. Clinical Trials for Treatment of Weakness of Muscles

These complexes are useful for treatment Of Myopathy. Chronic Fatigue Syndrome, Male Infertility, Stroke, Parkinsonism and Multiple Sclerosis.

The complex used in treatment of muscle weakness for different underlying causes (muscle dystrophies post stroke weakness). Clinical improvement in DMS patients is dose dependant affected by the age weight & degree of dystrophy. When the patient reaches the optimum dose, onset of improvement in muscle power is observed within hours of drug intake.

5. Clinical trials in Cardiomyopathy

The complex improved all cases of dilated Cardiomyopathy secondary to Duchenne muscle dystrophy, and 80% of cases of dilated cardiomyopathy due to other causes. Improvement is shown both clinically and in the ECHO cardiographic measurements.

6. Male Infertility

Our study included 20 patients: nine (9) patients azospermic, six (6) acino-terato-pyospermia, five (5) patients acino-oligo spermia, seven out of nine patients of azospermic count increased to (1.5-3 millions/hpf) within 6 weeks of treatment and two of them got healthy babies while the other two cases showed no response to treatment. Oligospermia showed doubling of the count within 1 month of treatment; four cases got also healthy babies. All cases showed significant improvement of motility.

7. Clinical Trials for Treatment of Fatigue in Humans

In all types of fatigue the complex relieved the muscle pain, increased the physical activity and productivity and associated depression. The drug improves markedly the agonizing fatigue in cases of Parkinsonism, myositis, disseminated sclerosis.

8. Mechanism of Action

The drug showed multiple system effects which suggests a common mechanism of action to link between effect on spermatogenesis, effect on muscle weakness, on immune system, and improving cardiac muscle in cardiomyopathy patients. We hypothesized that the drug may have a role in stimulating the stem cells present in the various organs to differentiate. Our hypothesis was proved by studying the effect of the drug both in vivo and in vitro on the stem cell differentiation. In vivo we measured the stem cell count together with CD8 and CD4 cells before, and at regular intervals of medication. There is a significant decrease in stem cell count with an increase in CD4 and CD8 number with no disturbance of CD4/CD8.

Copper is essential for the proper functioning of copper-dependant enzymes, including cytochrome C oxidase (energy production), superoxide dismutase (antioxidant protection), tyrosinase (pigmentation), dopamine hydroxylase (catecholamine production), lysyl oxidase (collagen and elastin formation), clotting factor v (blood clotting), and ceruloplasmin (antioxidant protection, iron metabolism, and copper transport).

The patentable embodiments of this invention which are claimed are as follows:

1. The copper(I) halide complex of pyrazinic acid having pharmaceutical activity.

2. The complex according to claim 1, comprising a pyrazinic acid-copper chloride complex.

3. The method for the preparation of the pyrazinic acid copper chloride complex, according to claim 2, the method comprising adding dropwise a solution of copper (I) chloride in an aqueous polar solvent, to a solution of pyrazinic acid, formed by dissolving the pyrazinic acid in boiling acetone (molar ratio=1:2.5), until a clear reddish solution is obtained; allowing the final mixture to stand and cool to deposit a micro-crystalline precipitate of the complex; filtering off the precipitate at a pump; washing the precipitate with acetone; and drying in vacuum, to form a dried precipitate.

4. The method for the preparation of the pyrazinic acid copper chloride complex, according to claim 3, wherein the aqueous polar solvent is selected from aqueous solutions of acetone and water-soluble alcohols

5. The pyrazinic acid-copper(I) chloride complex according to claim 2, having the empirical chemical formula: C₁₀H₁₂ClCuN₄O₆.

6. The pyrazinic acid copper(I) chloride complex according to claim 2, admixed with at least one pharmaceutically acceptable inert diluent.

7. The admixed pyrazinic acid copper(I) chloride complex according to claim 6 further comprising a pharmaceutically acceptable composition with other vitamins.

8. The admixed pyrazinic acid copper(I) chloride complex according to claim 6 in a solid dosage form suitable for oral administration selected from the group consisting of capsules, tablets, pills and granules.
